(19) United States
(12) Patent Application Publication
(10) Pub. No.: US 2009/0064474 A1
(43) Pub. Date: Mar. 12, 2009

(54) Bio-bones prosthetic system for tissue donor cadavers

(76) Inventor: Carol Jane Dancer, Attleboro, MA (US)

Correspondence Address: Carol Dancer;Absorbent Specialty Products, 413 Central Ave. # 10, Pawtucket, RI, 02861, US

(21) Appl. No.: 12/222,195

(22) Filed: Aug. 5, 2008

Related U.S. Application Data
(60) Provisional application No. 60/935,911, filed on Sep. 6, 2007.

(51) Int. Cl. A01N 1/00; A61F 2/28

(52) U.S. Cl. 27/21.1; 623/16.11

(57) ABSTRACT

The bio-bones prosthetics are designed to replace harvested bones from deceased tissue donors. Rigid bone replacement prosthetics offer structure and fill voids to provide a normal appearance to the donor. Each of the bio-bones prosthetics consist of rigid telescopic tubes, manufactured from bone colored polylactic acid, a biodegradeable material, making them ideal for both cremation and burial. They are extruded in slightly oval form, with the smaller units sliding inside the larger tubes, to provide continuous adjustment in length. A simple twist of the inner unit locks them in place. No foreign objects are need to secure them in place. This prevents any injuries to the users or cause any protrusions in the donors skin. The tubes are moldable, both prior to recovery and on-site allowing for a custom fit. Molded attachments are provided to secure the prosthetics in place.

### FIELD OF THE INVENTION

This present invention relates to donor reconstruction post recovery. The deceased donor will have had extensive surgical procedures to remove their bones. Bio-Bones prosthetics are provided to replace the recovered bones and create a normal appearance for a funeral service.

### BACKGROUND OF THE INVENTION

Extensive surgery is necessary to recover bones for tissue donation. In order to provide a funeral service for the donor, the deceased will need to be reconstructed and provided with rigid support to the remaining limp tissue.

Past solutions include broom sticks, PVC pipes and recently, wood and cardboard. All recent developments have an adjustable length & some form of foreign fastening device.

PVC pipes create toxic fumes during cremation.

Metal fastenings are can be sharp, and create a safety hazard for users.

Wooden pegs can be difficult to adjust, especially in moist areas.

Cardboard designs run the risk of softening when wet.

It is therefore an object of the invention to provide a family of bone replacement prosthetics for cadavers.

It is another object of the invention to provide continuously adjustable prosthetics to precisely fit the body of the donor.

It is another object of the invention to have these prosthetics easy and safe to use.

It is another object of the invention to provide environmentally friendly materials.

It is another object of the invention to not include any foreign materials.

It is another object of the invention that the material is readily moldable, even on site by user.

It is another object of the invention to provide a material that will biodegrade with the body in both cremation and burial.

### SUMMARY OF THE INVENTION

In accordance with the present invention, the bone replacement prosthetics include Upper Extremity for full arm, Humerus, Lower Extremity for full legs, Pelvic, Full Spine/Knee Block and Cervical/Lumbar. All units are continuously adjustable in length and a simple twist & lock motion locks them in place.

Continual adjustability allows for a perfect, secure fit, unlike with stepped adjustments, that limit the adjustments. There are no external locking devices to protrude through the skin. These features leave the limb with a more natural appearance. The muscular and other body fibers help to hold the prosthetics in place, and the lack of rigid fastening devices permits limb movement in the subsequent dressing process.

The prosthetics are readily moldable, both before shipment and on site, allowing for a more precise fit. In some cases, pre-molded attachments are supplied for a quick & accurate fit. These features are unique, and offer a custom fit.

In some cases a securing device is necessary to hold the prosthetic in place in the donor. Pre-molded end caps with a protruding peg, are provided to accommodate this need.

All prosthetics and pre-molded attachments are manufactured from sugar extracted from corn to make polylactic acid. This substance is readily extruded or molded, is rigid when cooled, but may be re-molded with boiling water. This is the same lactic acid that is found in muscle tissue of the human body, making them ideal for both burial and cremation. When cremation is selected for body disposal, no toxic gases will be released from these prosthetics.

The end result of these features is a family of cadaver bone replacement prosthetics that provide an accurate fit, rigidity to otherwise limp tissue, safe and easy to use and are both burial & cremation friendly. The result offers the donor a normal appearance for a funeral service.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a front view of an overview of a skeleton showing the bio-bone prosthetics replacing a full arm, two full legs, a section of the lumbar spinal column and the pelvic area;

FIG. 2 is a front view of an overview of a skeleton showing bio-bone prosthetics replacing the humerus on both arms, the lumbar spine, a full leg with molded femur head and ankle and a knee block on the other leg;

FIG. 3 is a perspective view of the oval section of each extrusion. demonstrating the twist locking mechanism;

FIG. 4 is a detail view of the separate bio-bone molded attachments, used in the assembly of different components; and

FIG. 5 is a detail view of the assembled bio-bone components, layed out in a manner corresponding to the position used in a donor.

For purposes of clarity and brevity, like elements and components will bear the same designations and numbering throughout the Figures.

### DESCRIPTION OF THE PREFERRED EMBODIMENT

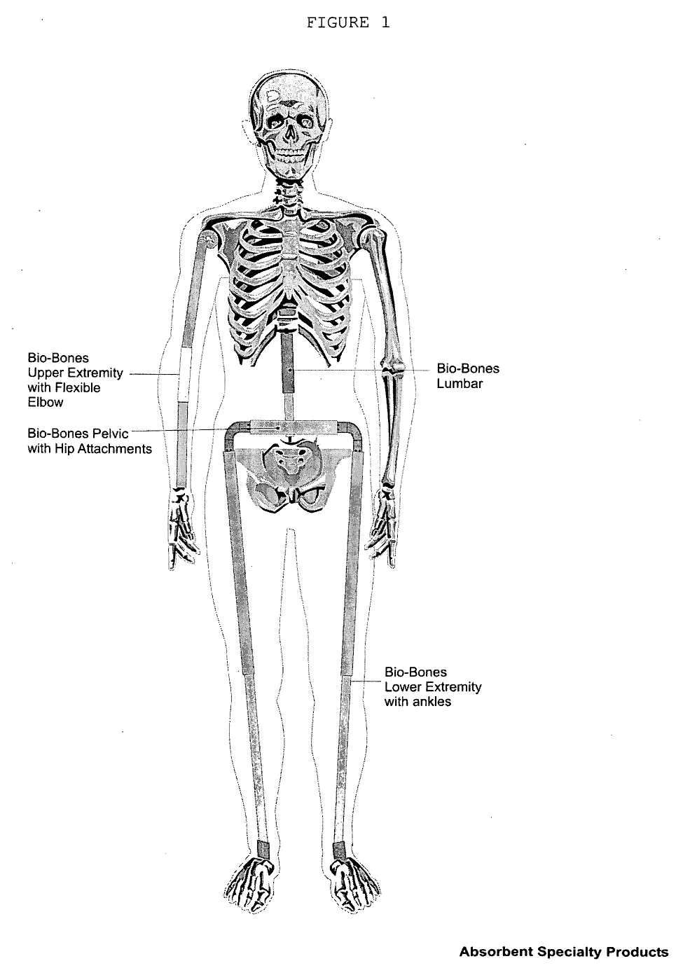

FIG. 1 is a frontal view of a skeleton of a cadaver. The skeleton shows bones that have been removed and replaced with the bio-bones prosthetics. The full upper extremity 18 prosthetic includes an upper humerus 10 with a hand molded 42 end to create a humerus 10 head to fit into the shoulder socket, a middle flexible elbow 16 to allow flexibility in movement and a lower portion that replaces the Radius & Ulna 48. All components are circular and fit together with a twisting action into the circular flexible tubing. Length is adjusted by twisting closer together to make the system shorter or twisting further apart to increase the length. Both rigid tubes have an abrasive surface 40 to allow adjustments without slippage.

A portion of the spinal column has been removed and replaced with the lumbar 36 prosthetic. This system includes a larger oval tube and a smaller oval tube that when fitted together can be adjusted in length by sliding to the desired length and then twisting them in opposite directions to squeeze the tubes together and provide a friction lock 46. Each of the exposed ends of the tubes is fitted with a press fit peg that leaves an exposed pin that is used to secure the system in place by fitting them into the cavity of the remaining bones. Both rigid tubes have an abrasive surface 40 to allow adjustments without slippage.

Both legs and the iliac crest have been removed and replaced with the lower extremity 24 prosthetics with molded ankle 28 and molded hip 22 attachments and the cross pelvic 20 prosthetic. The lower extremity 24 prosthetic includes two larger oval tubes 30 and two smaller oval tubes 30 that when fitted together can be adjusted in length by sliding to the desired length and then twisting them in opposite directions to squeeze the tubes together and provide a friction lock 46. Each of the smaller tubes is fitted with a press fit molded ankle 28 to fill the void from the removed talus & calcaneus. Each of the larger tubes is press fitted with a molded hip 22 attachment. Each of the hip attachments are then press fitted into the cross pelvic 20 to allow the entire system to be locked together. Both small and large rigid tubes in the lower extremity 24 prosthetics and the pelvic 20 have an abrasive surface 40 to allow adjustments without slippage.

All tubings and moldings are produced using a surgical blue colored biological material made of polylactic acid 34, thus providing a system that will degrade with the cadaver whether it is sent to burial or cremation and the blue color separates the Bio-Bones apart from other plastic prosthetics.

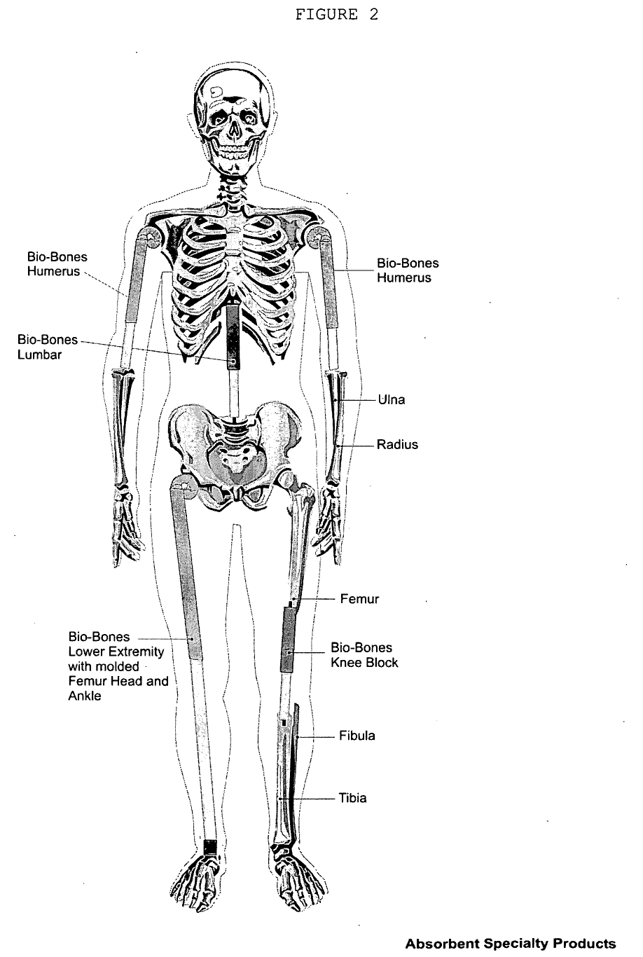

FIG. 2 is a frontal view of a skeleton of a cadaver. The skeleton shows bones that have been removed and replaced with the bio-bones prosthetics. Both upper arms have been replaced. These humerus 10 prosthetics include a larger oval tube and a smaller oval tube that when fitted together can be adjusted in length by sliding to the desired length and then twisting them in opposite directions to squeeze the tubes together and provide a friction lock 46. Each of the upper larger tubes have been hand molded 42 to create a humerus 10 head to fit into the shoulder socket. Both rigid tubes have an abrasive surface 40 to allow adjustments without slippage.

A portion of the spinal column has been removed and replaced with the lumbar 36 prosthetic. This system includes a larger oval tube and a smaller oval tube that when fitted together can be adjusted in length by sliding to the desired length and then twisting them in opposite directions to squeeze the tubes together and provide a friction lock 46. Each of the exposed ends of the tubes is fitted with a press fit peg that leaves an exposed peg that is used to secure the system in place by fitting them into the cavity in the ends of the remaining bones. Both rigid tubes have an abrasive surface 40 to allow adjustments without slippage.

The knee portion of the left leg has been removed and replaced with the knee block 38 prosthetic. This system includes a larger oval tube and a smaller oval tube that when fitted together can be adjusted in length by sliding to the desired length and then twisting them in opposite directions to squeeze the tubes together and provide a friction lock 46. Each of the exposed ends of the tubes is fitted with a press fit peg that leaves an exposed pin that is used to secure the system in place by fitting them into the cavity in the ends of the remaining bones. Both rigid tubes have an abrasive surface 40 to allow adjustments without slippage.

The right leg has been removed and replaced with the lower extremity 24 prosthetic with a molded femur head to fit into the hip socket and a molded ankle 28 attachment. The lower extremity 24 prosthetic includes a larger oval tube and a smaller oval tube that when fitted together can be adjusted in length by sliding to the desired length and then twisting them in opposite directions to squeeze the tubes together and provide a friction lock 46. The smaller tube is fitted with a press fit molded ankle 28 to fill the void from the removed talus & calcaneus. The end of the larger tube has been hand molded 42 to fit into the hip socket. Both small and large rigid tubes in the lower extremity 24 prosthetic have an abrasive surface 40 to allow adjustments without slippage.

All tubings and moldings are produced using a surgical blue colored biological material made of polylactic acid 34, thus providing a system that will degrade with the cadaver whether it is sent to burial or cremation and the blue color separates the Bio-Bones apart from other plastic prosthetics.

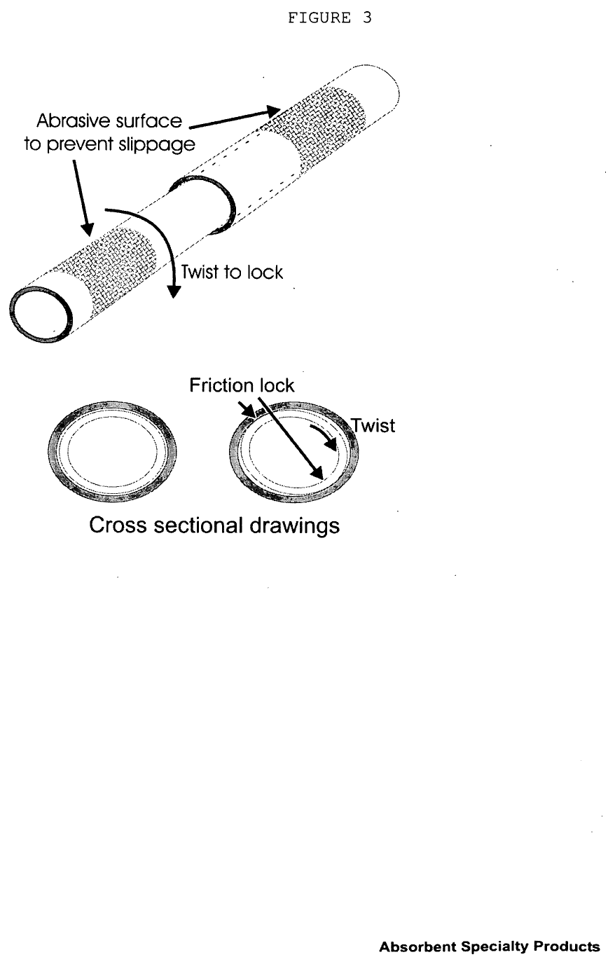

FIG. 3 is a perspective view of the oval tubes 30 section of each extrusion. The two extrusions are designed to have the smaller size slide inside of the larger size. When the units are twisted, relative to each other, the oval shape causes the two pieces to bind against each other, and the resulting friction locks them in place at the selected length. Each tube is supplied with an abrasive surface 40 to prevent any slippage during adjustments. The cross sectional drawing demonstrates the friction lock 46 that occurs during the twisting action.

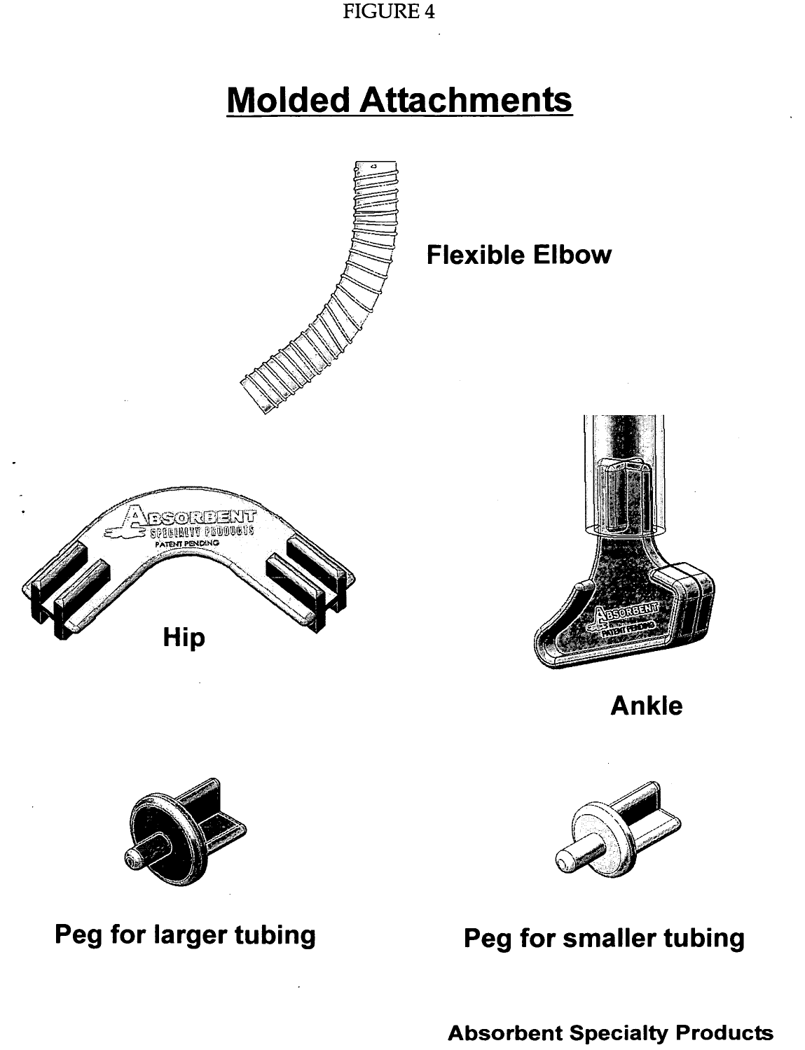

FIG. 4 is a detail of each of the attachments. The flexible elbow 16 shows the helix design that allows the round rigid tubing 44 to be twisted into place and secured with a simple friction fit in the full upper extremity 18. The molded hip 22 attachment shows how the “H” ends press fit into the oval tubing to prevent them from twisting and will hold the attachment in only one direction in both the upper tubing of the lower extremity 24 and the cross pelvic 20. The molded ankle 28 attachment has a “x” end that allows simple twisting to assure that it lines up correctly with the foot and fits into the lower tubes of the lower extremity 24. Large molded peg 1 50 and molded peg 3 54 fits into the larger oval tube using an “x” to hold it in place and provides a peg to secure into the cavity space in the remaining bones and is used in the lumbar 36, cervical 26, spine 14 and knee block 38. Small molded peg 2 52 and molded peg 4 56 fit into the smaller oval tube using an “x” to hold it in place and provides a peg to secure into the cavity space in the remaining bones and is used in the lumbar 36, cervical 26, spine 14 and knee block 38.

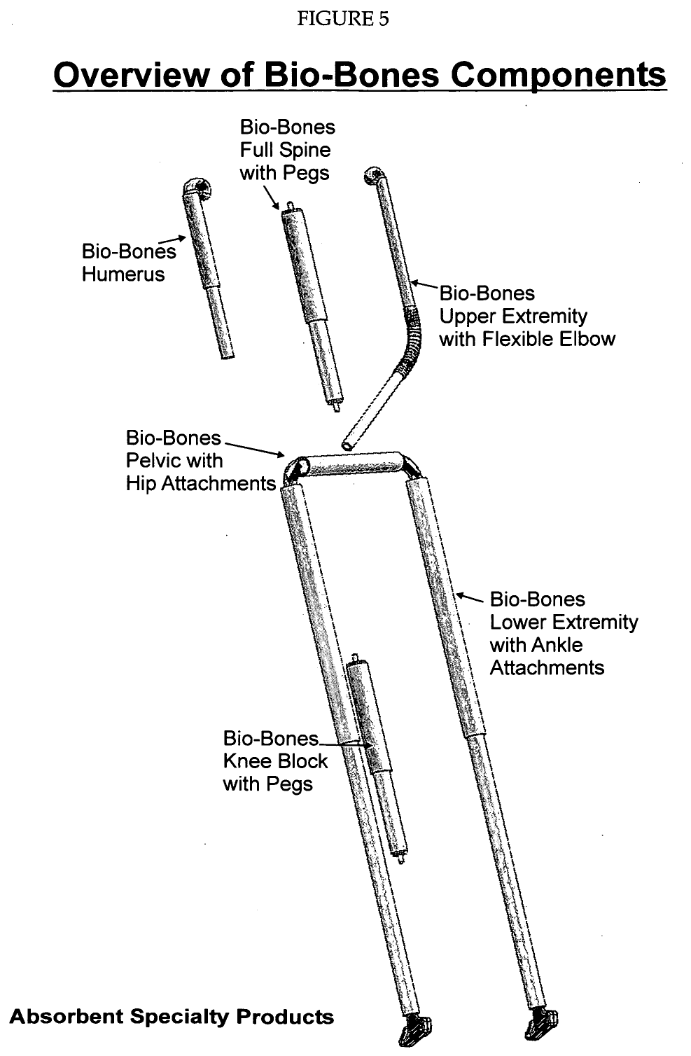

FIG. 5 is a frontal view of bio-bones prosthetics that comprise this patent application. This view displays the prosthetics as they would be placed in the donor.

The right arm humerus 10 prosthetic displays the replacement of the upper arm. The humerus 10 is supplied with a hand molded 42 end to fit the shoulder socket and an abrasive surface 40 for securing in place.

The left arm displays the full upper extremity 18 with flexible elbow 16. The full upper extremity 18 is supplied with a hand molded 42 end to fit the shoulder socket and an abrasive surface 40 for securing in place.

The full spine 14 demonstrates a replacement of the spinal column. The spine 14 prosthetic is provided with a large molded peg 3 54 and a small molded peg 4 56 to fit in each end and an abrasive surface 40 for securing in place.

The knee block 38 displayed next to the full lower extremity 24 to show the versatility of using individual components, may also be used in an amputee recovery. The knee block 38 is supplied with a large molded peg 3 54 and a small molded peg 4 56 to fit in each end and an abrasive surface 40 for securing in place.

The full lower extremity 24 is displayed in position with the pelvic 20 attached for an iliac crest recovery. The full lower extremity 24 is provided with ankles to fit in the lower tubes and an abrasive surface 40 for securing in place.

The pelvic 20 prosthetic is supplied with two molded hip 22 attachments to connect to both the upper tubes of the lower extremity 24 and the pelvic 20 and an abrasive surface 40 for securing in place.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A bio-bones prosthetic system for tissue donor cadavers for replacing bones to reconstruct the body to a normal appearance for a funeral service, comprising:
a rigid tubing, for providing structure to the donor once bones have been recovered;
an oval tubes, for providing a simple way to adjust the length of each component and providing a simple twist locking mechanism that offers ease of use & function with no need for any sharps, pins, pegs, ties or screws;
a friction lock, for providing a simple way to lock tubes in place at the desired length;
a polylactic acid, for providing the funeral home with ease of processing the donor so that the body will degrade in either burial or cremation;
a hand molded, for offering a custom fit prior to, or during recovery. rigid tubes can be molded onsite using boiling water and re-adjusted to fit each donor;
an abrasive surface, for creating a friction surface to minimize any slippage during adjustments;
an upper extremity, for replacing recovered humerus, radius and ulna with a flexible elbow;
a humerus, for replacing a recovered humerus bone;
a flexible elbow, for allowing movement in the elbow, adjustably connected to said humerus;
a radius & ulna, for replacement of the lower arm, adjustably connected to said flexible elbow;
a lower extremity, for replacing recovered femur, tibia & fibula;
a molded ankle, for attaching into the lower extremities to provide ankle support, rigidly connected to said lower extremity;
a pelvic, for connecting the lower extremities after hemi pelvis recovery;
a molded hip, for attaching to both the pelvic and lower extremity prosthetics to lock components in place, rigidly connected to said pelvic, and rigidly connected to said lower extremity;
a spine, for replacing a recovered portion of the spinal column;
a cervical, for replacing recovered portions of the upper spinal column;
a lumbar, for replacing recovered portions of the lower spinal column;
a knee block, for replacing recovered knee from either leg;
a peg 1, for use in securing upper portion of bio-bone lumbar or knee block into the remaining cavity in the bones, rigidly connected to said lumbar;
a peg 2, for use in securing lower portion of bio-bone lumbar or knee block into the remaining cavity in the bones, rigidly connected to said lumbar;
a peg 3, for use in securing upper portion of bio-bone lumbar or knee block into the remaining cavity in the bones, rigidly connected to said knee block; and
a peg 4, for use in securing lower portion of bio-bone lumbar or knee block into the remaining cavity in the bones, rigidly connected to said knee block.
